(19) United States
(12) Patent Application Publication
(10) Pub. No.: US 2009/0274633 A1
(43) Pub. Date: Nov. 5, 2009

(54) TOOTH POWDER CONTAINING ELVAN AS RADIATION SOURCE OF FAR INFRARED RAY

(76) Inventor: Jae-Hong Song, Guansan-si (KR)

Correspondence Address: MERCHANT & GOULD PC, P.O. BOX 2903, MINNEAPOLIS, MN, 55402-0903, US

(21) Appl. No.: 12/112,468

(22) Filed: Apr. 30, 2008

(51) Int. Cl. A61K 8/18

(52) U.S. Cl. 424/49

(57) ABSTRACT

The present invention relates to tooth powder containing elvan as a radiation source of a far infrared ray wherein the tooth powder includes 10 weight % to 90 weight % of elvan as a radiation source of a far infrared ray and 10 weight % to 90 weight % of bamboo salt, and is characterized in that the rate of the elvan and the bamboo salt is 10:90 to 90:10. Also, it is of sovereign remedy in preventing teeth from decaying with medicine made by desiccating sap of a lacquer tree.

### BACKGROUND OF THE INVENTION

1. Field of Invention

The present invention relates to tooth powder containing elvan as a radiation source of a far infrared ray, more particularly, to tooth powder containing elvan as a radiation source of a far infrared ray wherein the tooth powder includes 10 weight % to 90 weight % of elvan as a radiation source of a far infrared ray and 10 weight % to 90 weight % of bamboo salt, and is characterized in that the rate of the elvan and the bamboo salt is 10:90 to 90:10.

2. Description of the Related Art

As known in the related art, the conventional toothpaste which contains peroxides for the beautifying effect and an abrasive for improving an abrasive property accommodates the beautifying effect by physically drilling a tooth surface. However, such toothpaste has problems of causing hypersensitive symptoms in teeth and abrasion states in the gingival region by excessive wearing of the teeth due to the use of long period as well as it has no more effect than expected.

Further, the abrasive such as calcium carbonate, alumina, silica gel, pyrophosphoric acid calcium, calcium phosphate, and bicarbonate of natrium stimulates a mucous membrane of the mouth to cause a blister, erythema, and bulge, with the preservative contained in the toothpaste like para-oxy benzoic acid methyl, para-oxy benzoic acid propyl. Also, it is required to take counsel with a doctor or a pharmacist, when swallowing it by a mere accident. Especially, in case of conventional tooth powder used for erasing bacterial membrane and plague, directions for the use of medicine such as “Do not use for children under six years of age. In case of using for children over seven years old, use under the guidance of a protector. Take counsel with a doctor or pharmacist when swallowing by accent” are specified in the instruction book of a product.

### SUMMARY OF THE INVENTION

Accordingly, the present invention is directed to tooth powder containing elvan as a radiation source of a far infrared ray that substantially obviates one or more problems due to limitations and disadvantages of the related art.

The object of the present invention is to provide tooth powder containing elvan as a radiation source of a far infrared ray wherein the tooth powder includes 10 weight % to 90 weight % of elvan as a radiation source of a far infrared ray and 10 weight % to 90 weight % of bamboo salt, and is characterized in that the rate of said elvan and bamboo salt is 10:90 to 90:10.

Another object of the present invention is to provide tooth powder containing elvan as a radiation source of a far infrared ray wherein the tooth powder sterilizes in the mouth, prevents propagation of bacilli, excludes ozostomia, maintains continuous feeling of purity over 12 hours, prevents bleeding from the teethridge, removes tartar and nicotine of teeth, and suppresses joggling of teeth, using the function of the elvan powder.

Still, another object of the present invention is to provide tooth powder containing elvan as a radiation source of a far infrared ray wherein the radiation of the far infrared ray from elvan promotes the circulation of the blood, excludes ozostomia, and executes the functions of anti-bacteria, anti-phlogist, and abrasion, besides accommodates abrasive action with the antibiotic bamboo salt.

Additional advantages, objects, and features of the invention will be set forth in part in the description which follows and in part will become apparent to those having ordinary skill in the art upon examination of the following or may be learned from practice of the invention.

The objectives and other advantages of the invention may be realized and attained by the structure particularly pointed out in the written description and claims hereof.

### DETAILED DESCRIPTION OF THE INVENTION

The elvan used for a radiation source of a far infrared ray in the present invention is “a grain of boiled rice of barley” shaped rock where red spots and white spots are closely stuck in the stone of a yellowish white color, a light yellowish brown color or a light color, according to the “Dong-eu-bo-gam” (Eastern medical thesaurus) written by Hur Jun(Oriental medical doctor) and “Bon-cho-gang-mok” (Botanical list) from China, it was used for an anti-phlogistic agent to the various skin diseases like a boil, an abscess on one's back due to its warm and sweet properties without toxicity, and named medicine and acupuncture that is a mysterious stone in the Dong-eu-bo-gam.

Elvan which belongs to quartz porphyry among igneous rocks contains silicon dioxide SiO₂ and aluminum oxide Al₂O₃ as main components, and contains Fe₂O₃ needed for a human body and living cell as well as various mineral elements more than forty sorts, including many apertures of three hundred thousand to fifteen hundred thousand per one cm³. Also, elvan described above executes the function such as a growth of living body, control of physiology function, maintaining of vitality in the cell, prophylaxis and remedy of various diseases, counteracting poison in human body and promotion of rearing. Such effects of the elvan can be easily found in one's real life, for example, an aquarium, a fish bowl, and a fish farm using the elvan attains the effect of water purity improvement with outstanding water-purity control function.

Also, in elvan having five functions, the decomposition action of a heavy metal as its first function is that it adsorbs and dissolves a heavy metal and a pollutant utilizing a capillary phenomenon due to its wide surface area including multi-layers and multi-holes more than three hundred thousand per 1 cm³. The strong adsorbent property of elvan originates from that its main components are silicon dioxide and aluminum oxide and the kaolin state by weathering a part of the feldspar and great wide surface area by the porosity.

As a result of testing the adsorbent property of elvan actually, it represented 85% of adsorbent result for 4 hours when injecting 10% elvan in the mercury solution of 0.1 ppm, and 43% of adsorbent result after 3 hours when injecting 10% elvan in the cadmium solution of 0.1 ppm. Also, in case of a bacillus, 56 to 60% of bacilli were adsorbed to the stone and removed as a result of passing white staphylococcus solution through the elvan layer of approximately 10 mesh for 10 minutes.

Secondly, the mineral eruption action where about 40 sorts of minerals giving vitality to the human body are dissolved improves metabolism and skin health. Most of rocks contain the mineral elements, still the elvan contains the mineral known to the public as well as erupts effective minerals unexplained by the modern science.

Thirdly, the water-purity control action is that elvan controls the quality of water in the water. Namely, it changes hard water into soft water (softening). Such action softens hard water into soft water by ion-exchanging the feldspar as main element of elvan with calcium ions or magnesium ions.

Fourthly, as sufficient oxygen content, injecting elvan into the water causes drop of biological oxygen demand (BOD) and chemical oxygen demand (COD) for giving vitality to the living body with preserving action.

Fifthly, as the action of radiating and emitting a far infrared ray, elvan maintains the degree of freshness in food due to resonance and absorption function by radiating a far infrared ray, and promotes the circulation of the blood. The far infrared ray that elvan radiates penetrates into the inner portion 40 to 50 mm away from the skin to activate the cells for promoting the circulation of the blood and for improving natural healing power by discharging wastes from the body.

On the other hand, a far infrared ray having 30 to 1000 μm of wave-length penetrates into the hypodermic in-depth eighty times deeper than general heat when absorbing in the human body to radiate in the protein molecules constituting the body cell for activating the cells by minutely vibrating the cells approximately 2000 turns per 1 minute. Such cellular activation promotes living activity of the human body. Further, such overall action causes heat energy and discharges wastes of cells from the body naturally.

Likewise, it is known that the far infrared ray affects the cell as basic unit of the human body to promote the circulation of the blood for strengthening defense capability of the human body, besides executes the function of deodorization, mothproof, and palliation of a pain.

The theory about various actions of the far infrared ray described until now may be clearly seen in the far infrared ray treatment originated by Yamazaki Dosiko in Japan who is a doctor of medicine/science as well as medical researcher.

The tooth powder containing elvan as a radiation source of a far infrared ray according to the present invention includes 10 weight % to 90 weight % of elvan as a radiation source of a far infrared ray and 10 weight % to 90 weight % of bamboo salt, and is characterized in that the rate of said elvan and bamboo salt is 10:90 to 90:10.

Also, it further includes xylitol 1 to 10 parts by weight as natural sweetener, D-sorbitol 1 to 5 parts by weight as a wetting agent, and mugwort powder 1 to 5 parts by weight, on the basis of tooth powder 100 parts by weight.

It is preferable to include bamboo salt over 10 weight % for using as an abrasive.

Further, the tooth powder containing elvan as a radiation source of a far infrared ray is characterized by further including pine resin powder 1 to 10 parts by weight.

Also, the tooth powder containing elvan as a radiation source of a far infrared ray includes 10 weight % to 90 weight % of elvan as a radiation source of a far infrared ray and 10 weight % to 90 weight % of burnt salt, and the rate of said elvan and burnt salt may be 10:90 to 90:10.

One of ginseng steamed red, ginseng, herb, and licorice powder as a wetting agent may be further included in 1 to 5 parts by weight, on the basis of tooth powder 100 parts by weight.

It is preferable that diameter of bamboo salt powder and elvan as a radiation source of a far infrared ray has proper particle. That is because the abrasive effect is reduced when a diameter of a particle is smaller than said extent, and a feeling of an alien substance is felt in using when the diameter is larger than this.

Moreover, medicine made by desiccating sap of a lacquer tree may be included, and the medicine is used for curing of a cough, an intestinal disease, an emmenagogue, and killing insects etc., still the effect of preventing teeth from decaying may be gained in case of adding to the tooth powder by a powder type.

In addition to that, it takes effect on curing for the disease of the teethridge, in case of adding a desiccated safflower within the chrysanthemum family and a kind of rhubarb as a root in the rhubarb within the knotgrass family. As added amount, 1 to 5 parts by weight may be added to that, on the basis of total 100 parts by weight.

As described above, the present invention provides tooth powder containing elvan as a radiation source of a far infrared ray wherein the tooth powder sterilizes in the mouth, prevents propagation of bacilli, excludes ozostomia, maintains continuous feeling of purity over 12 hours, prevents bleeding from the teethridge, removes tartar and nicotine of teeth, and suppresses joggling of teeth.

Although a few exemplary embodiments of the present invention have been shown and described, it will be appreciated by those skilled in the art that changes may be made in these embodiments without departing from the principles and spirit of the invention, the scope of which is defined in the appended claims and their equivalents.

What is claimed is:

1. Tooth powder containing elvan as a radiation source of a far infrared ray, wherein the tooth powder includes 10 weight % to 90 weight % of elvan as a radiation source of a far infrared ray and 10 weight % to 90 weight % of bamboo salt, and is characterized in that the rate of the elvan and the bamboo salt is 10:90 to 90:10.

2. The tooth powder containing elvan as a radiation source of a far infrared ray according to claim 1, wherein the tooth powder further includes xylitol 1 to 10 parts by weight as natural sweetener, D-sorbitol 1 to 5 parts by weight as a wetting agent, and mugwort powder 1 to 5 parts by weight, on the basis of tooth powder 100 parts by weight.

3. The tooth powder containing elvan as a radiation source of a far infrared ray according to claim 1, wherein the tooth powder further includes pine resin powder 1 to 10 parts by weight on the basis of tooth powder 100 parts by weight.

4. Tooth powder containing elvan as a radiation source of a far infrared ray includes 10 weight % to 90 weight % of elvan as a radiation source of a far infrared ray and 10 weight % to 90 weight % of burnt salt, and the rate of said elvan and burnt salt may be 10:90 to 90:10.

5. The tooth powder containing elvan as a radiation source of a far infrared ray according to claim 1, wherein one of ginseng steamed red, ginseng, herb, and licorice powder as a wetting agent may be further included in 1 to 5 parts by weight on the basis of tooth powder 100 parts by weight.

6. The tooth powder containing elvan as a radiation source of a far infrared ray according to claim 1, wherein the tooth powder further includes medicine made by desiccating sap of a lacquer tree 1 to 5 parts by weight on the basis of tooth powder 100 parts by weight.
